(19) United States
(12) Patent Application Publication
(10) Pub. No.: US 2002/0006412 A1
(43) Pub. Date: Jan. 17, 2002

(54) Preparation and use of particulates composed of adenovirus particles

(76) Inventors: Bruce L. Roberts, Southboro, MA (US); Charles Nicolette, Framingham, MA (US); Srinivas Shankara, Shrewsbury, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 09/841,836

(22) Filed: Apr. 25, 2001

Related U.S. Application Data
(60) Provisional application No. 60/200,195, filed on Apr. 28, 2000.

(51) Int. Cl. A61K 39/235; A61K 39/23; C12N 7/01

(52) U.S. Cl. 424/233.1; 435/235.1

(57) ABSTRACT

This invention provides particulates of adenoviral particles comprised of individual adenovirus virions complexed to an insoluble micro-platform material and for such compositions further comprised of a polynucleotide encoding an antigenic peptide. The invention further provides method for forming such complexes such that the compositions are useful for transfecting phagocytic antigen presenting cells such as dendritic cells and for vaccinating a subject against disease.

### TECHNICAL FIELD

The present invention relates to the fields of molecular biology and immunology and more specifically is directed to compositions and methods for vaccinating against disease.

### BACKGROUND OF THE INVENTION

Recombinant viruses have been shown to be useful as vaccine vectors and adenoviruses in particular have been developed to introduce genes encoding antigenic proteins in order to stimulate immune responses (Graham and Prevec (1992) Biotechnology 20:363-390; Imler (1995) Vaccine 13(13):1143-1151). At the same time, adenovirus vectors have been used extensively to develop gene delivery technology for gene therapy applications (Kozarsky and Wilson (1993) Curr. Opin. Genet. Dev. 3(3):499-503). As a result, there is a large knowledge base regarding application of adenovirus technology for gene delivery and vaccination against disease. Nevertheless, while these vectors have been found to be effective for introducing a variety of genes into multiple different cell types, both replicating and non-replicating, the efficiency of transfection as well as the tendency of such vectors to infect cells other than the selected target cells has prevented the full utilization of this promising technology.

At the same time that technology for construction of recombinant vaccines and gene delivery vehicles has been progressing, basic understanding of immunity and the functions of multiple interacting components of the immune system has also advanced significantly. Details of the processes which govern the establishment of humoral and cell mediated immune responses have been elucidated at the molecular and cellular level and the mechanisms of antigen presentation and recognition have been substantially explained. As a result, the importance of antigen presenting cells and especially dendritic cells in inducing an effective immune reaction are now appreciated

Dendritic cells are the most potent antigen presenting cells in the body, being able to present antigenic peptides in the context of both MHC class I and MHC class II molecules to CD8+ and CD4+ T cells respectively. There has been intense interest in the delivery of antigenic protein, peptides, and genes encoding these respective proteins and peptides to dendritic cells. Of these approaches, genetically modified dendritic cells appear to be superior to either peptide or protein pulsed cells in their ability to stimulate T cell responses, possibly due to the fact that genetically modified dendritic cells possess a renewable supply of antigenic peptides for presentation. Induction of immune responses to tumor antigens has now been demonstrated using adenovirus vectors to transfect dendritic cells, raising the possibility of developing effective vaccines against various forms of cancer (Kaplan et al., (1999) J Immunol. 163(2):699-707).

Adenoviral vectors have been shown to be a useful means to genetically modify dendritic cells and one can achieve 90% transduction of dendritic cells in vitro provided a multiplicity of infection of >100 and a suitably long incubation period are employed (Zhong et al., (1999) Eur. J. Immunol. 29(3):964-972). It has been found that the efficiency of adenoviral vector mediated gene transfer to dendritic cells improves with the duration of exposure of dendritic cells to adenovirus suggesting that the infection process is not instantaneous and that longer incubation periods favor greater uptake of adenoviral particles by dendritic cells. If the adenovirus vaccine is intended for use in vivo, the constraints required to achieve these relatively high transfection rates present a major technical challenge.

The uptake of adenovirus particles by infected cells has been shown to be mediated by binding to cell surface receptors such as the CAD receptor (Coxsackie adenovirus receptor). In addition, it has been shown that adenoviruses can be delivered to dendritic cells in vitro by attaching them to a substrate molecule capable of binding to alternative receptors that are highly expressed on dendritic cell surfaces (Tillman et al. (1999) J. Immunol. 162(11):6378-6383; Diebold et al., (1999) J. Biol. Chem. 274(27):19087-19094). In addition, such receptor mediated gene delivery can also be utilized to enhance the uptake of independent DNA vectors as has been seen historically with adenovirus assisted, receptor mediated gene delivery (Curiel et al., (1991) PNAS 88(19):8850-8854; Cotton et al., (1992) PNAS 89(13):6094-6098). Nevertheless, while such gene delivery can be performed in vitro, administration of similar compositions as vaccines in vivo presents serious technical difficulties.

It would be desirable to have a method for transfecting dendritic cells that could achieve high levels of transfection efficiency and that could be administered effectively in vivo as well as in vitro. Dendritic cells possess an intrinsic ability to engulf particulate material via the process of phagocytosis. This can readily be monitored by the addition of fluorescently labeled microbeads to dendritic cells. This natural propensity of dendritic cells to act as scavengers is in keeping with their central role in immune surveillance. The present invention seeks to enhance the efficiency of adenovirus mediated gene transfer to dendritic cells by capitalizing on the ability of dendritic cells to engage in endocytosis.

### DESCRIPTION OF THE INVENTION

Vaccination against disease by delivery of antigen encoding polynucleotides inserted into viral vectors has the potential to provide effective therapies for a variety of disease conditions if the polypeptides encoded by these polynucleotides can be effectively presented to immune effector cells. The present invention provides compositions and methods to achieve this purpose.

This invention provides a composition comprised of a plurality of adenovirus particles complexed to an insoluble micro-platform material. The particulate may be further comprised of a cell binding ligand to enhance delivery to antigen presenting cells, especially dendritic cells. In addition, the adenovirus particulate may be further comprised of a polynucleotide encoding an antigenic polypeptide.

The invention also provides methods for forming adenoviral particulates by complexing a plurality of adenovirus particles with an insoluble micro-platform material. Such complexes are formed by attachment with a crosslinking agent with reacts with both the adenovirus particles and the micro-platform material. The method of forming adenovirus particulates may also further comprise inclusion of a cell binding ligand suitable for directing attachment of the complex to an antigen presenting cell.

The compositions of the present invention are useful for delivering antigens and for vaccinating a subject against disease, thus the present invention provides methods for transfecting dendritic cells by contacting them with the adenovirus particulate and for vaccinating a subject against disease by administering to a subject the compositions of the invention.

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, MOLECULAR CLONING: A LABORATORY MANUAL, SECOND EDITION (Sambrook et al., 1989); CURRENT PROTOCOLS IN MOLECULAR BIOLOGY (F. M. Ausubel et al., eds., 1987); OLIGONUCLEOTIDE SYNTHESIS (M. J. Gait, ed., 1984); ANIMAL CELL CULTURE (R. I. Freshney, ed., 1987); METHODS IN ENZYMOLOGY (Academic Press, Inc.); HANDBOOK OF EXPERIMENTAL IMMUNOLOGY (D. M. Wei & C. C. Blackwell, eds.); GENE TRANSFER VECTORS FOR MAMMALIAN CELLS (J. M. Miller & M. P. Calos, eds., 1987); PCR: THE POLYMERASE CHAIN REACTION, (Mullis et al., eds., 1994); CURRENT PROTOCOLS IN IMMUNOLOGY (J. E. Coligan et al., eds., 1991); ANTIBODIES: A LABORATORY MANUAL (E. Harlow and D. Lane eds. (1988)); PCR 2: A PRACTICAL APPROACH (M. J. MacPherson, B. D. Hames and G. R. Taylor eds. (1995)) and ANIMAL CELL CULTURE (R. I. Freshney, ed. (1987)).

Definitions

As used herein, certain terms may have the following defined meanings.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous nucleotides.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989) Supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein with adenovirus vectors.

A “viral vector” is defined as a recombinantly produced virus or viral particle that comprises a polynucleotide to be delivered into a host cell, either in vivo, ex vivo or in vitro. Examples of viral vectors include adenovirus vectors, adeno-associated virus vectors, retroviral vectors and the like. In aspects where gene transfer is mediated by an adenoviral vector, a vector construct refers to the polynucleotide comprising the adenovirus genome or part thereof, and a therapeutic gene. As used herein, “adenoviral mediated gene transfer” or “adenoviral transduction” carries the same meaning and refers to the process by which a gene or nucleic acid sequences are stably transferred into the host cell by virtue of the virus entering the cell and expressing its genome within the host cell. While the virus has the ability to enter the host cell via its normal mechanism of the compositions of the present invention are particularly useful for infecting phagocytic antigen presenting cells by facilitating the process of engulfment.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. (see, e.g., WO95/27071). Ads are easy to grow and do not require integration into the host cell genome. Recombinant Ad-derived vectors, particularly those that reduce the potential for recombination and generation of wild-type virus, have also been constructed. (see WO95/00655 and WO95/11984). Wild-type AAV has high infectivity and specificity integrating into the host cell's genome. Hermonat and Muzyczka (1984) Proc. Natl. Acad. Sci. 81:6466-6470 and Lebkowski et al., (1988) Mol. Cell. Biol. 8:3988-3996.

“Adenovirus particulates” refers to complexes of adenovirus particles immobilized on or within a micro-polymeric matrix, fiber, microbead, or other solid micro-platform material. Such particulates are characteristically insoluble in aqueous solutions and comprised of a plurality of adenovirus particles complexed with an insoluble micro-micro-platform material.

“Adenovirus particles” are individual adenovirus virions comprised of an external capsid and internal nucleic acid material, where the capsid is further comprised of adenovirus envelope proteins. The adenovirus envelope proteins may be modified to comprise a fusion polypeptide which contains a polypeptide ligand covalently attached to the viral protein.

The term “micro-platform material” refers to a solid, insoluble substance which comprises a particle of suitable dimensions so that it can be engulfed by a phagocytic cell such as an antigen presenting cell, and in particular, a dendritic cell. The term is meant to include a variety of substances including but not limited to polymeric materials capable of forming into fibers, beads, or matrices, and capable for complexing with adenovirus particles via covalent or non-covalent bonds; such as hydophobic, hydophilic, ionic, or electrostatic attraction bonds.

The term “cross-linking agent” is meant to describe a reagent that can bind to both adenovirus particles and a micro-platform material so as to attach a plurality of adenovirus particles to the micro-platform. For example, the cross-linking agent can be a bifunctional antibody that binds to both virus and micro-platform or any other reagent with similar dual binding capacity.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6 X SSC to about 10 X SSC; formamide concentrations of about 0% to about 25%; and wash solutions of about 6 X SSC. Examples of moderate hybridization conditions include: incubation temperatures of about 40° C. to about 50° C.; buffer concentrations of about 9 X SSC to about 2 X SSC; formamide concentrations of about 30% to about 50%; and wash solutions of about 5 X SSC to about 2 X SSC. Examples of high stringency conditions include: incubation temperatures of about 55° C. to about 68° C.; buffer concentrations of about 1 X SSC to about 0.1 X SSC; formamide concentrations of about 55% to about 75%; and wash solutions of about 1 X SSC, 0.1 X SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Target cell” or “recipient cell” is intended to include any individual cell or cell culture which can be or have been recipients for vectors or the incorporation of exogenous nucleic acid molecules, polynucleotides and/or proteins and which are the target of lysis by the invention methods. It also is intended to include progeny of a single cell, and the progeny may not necessarily be completely identical (in morphology or in genomic or total DNA complement) to the original parent cell due to natural, accidental, or deliberate mutation.

The term “antigen” is well understood in the art and includes substances which are immunogenic, i.e., immunogens, as well as substances which induce immunological unresponsiveness, or anergy, i.e., anergens.

The term “immune effector cells” refers to cells capable of binding an antigen and which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates. Certain diseased tissue expresses specific antigens and CTLs specific for these antigens have been identified. For example, approximately 80% of melanomas express the antigen known as GP-100.

The term “immune effector molecule” as used herein, refers to molecules capable of antigen-specific binding, and includes antibodies, T cell antigen receptors, and MHC Class I and Class II molecules.

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected or measured, after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody).

The terms “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of a chain encoded in the MHC associated noncovalently with b2-microglobulin. Class I MHC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of noncovalently associated α and β chains. Class II MHC are known to participate in antigen presentation to CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a self class I or class II MHC molecule. Methods of identifying and comparing MHC are well known in the art and are described in Allen, M. et al. (1994) Human Immunol. 40:25-32; Santamaria, P. et al. (1993) Human Immunol., 37:39-50 and Hurley, C. K. et al. (1997) Tissue Antigens 50:401-415.

The term “antigen presenting cells (APCs)” refers to a class of cells capable of presenting one or more antigens in the form of antigen-MHC complex recognizable by specific effector cells of the immune system, and thereby inducing an effective cellular immune response against the antigen or antigens being presented. While many types of cells may be capable of presenting antigens on their cell surface for T-cell recognition, only professional APCs have the capacity to present antigens in an efficient amount and further to activate T-cells for cytotoxic T-lymphocyte (CTL) response. APCs can be obtained from a variety of cell types such as macrophages, B-cells and dendritic cells (DCs).

The term “dendritic cells (DCs)” refers to a diverse population of morphologically similar cell types found in a variety of lymphoid and non-lymphoid tissues (Steinman (1991) Ann. Rev. Immunol. 9:271-296). Dendritic cells constitute the most potent and preferred APCs in the organism. A subset, if not all, of dendritic cells are derived from bone marrow progenitor cells, circulate in small numbers in the peripheral blood and appear either as immature Langerhans' cells or terminally differentiated mature cells. While the dendritic cells can be differentiated from monocytes, they possess distinct phenotypes. For example, a particular differentiating marker, CD14 antigen, is not found in dendritic cells but is possessed by monocytes. Also, dendritic cells are not phagocytic, whereas the monocytes are strongly phagocytosing cells. It has been shown that DCs provide all the signals necessary for T cell activation and proliferation.

“Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz R. H. (1990) Science 248:1349-1356 and Jenkins M. K. (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs. Several molecules have been shown to enhance co-stimulatory activity. These are heat stable antigen (HSA) (Liu Y. et al. (1992) J. Exp. Med. 175:437-445), chondroitin sulfate-modified MHC invariant chain (Ii-CS) (Naujokas M. F. et al. (1993) Cell 74:257-268), intracellular adhesion molecule 1 (ICAM-1) (Van Seventer G. A. (1990) J. Immunol. 144:4579-4586), B7-1, and B7-2/B70 (Schwartz R. H. (1992) Cell 71:1065-1068). These molecules each appear to assist co-stimulation by interacting with their cognate ligands on the T cells. Co-stimulatory molecules mediate co-stimulatory signal(s) which are necessary, under normal physiological conditions, to achieve full activation of naïve T cells. One exemplary receptor-ligand pair is the B7 co-stimulatory molecule on the surface of APCs and its counter-receptor CD28 or CTLA-4 on T cells (Freeman et al. (1993) Science 262:909-911; Young et al. (1992) J. Clin. Invest. 90: 229 and Nabavi et al. (1992) Nature 360:266-268). Other important co-stimulatory molecules are CD40, CD54, CD80, CD86. The term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter, Inc. (Fullerton, Calif.). It is intended, although not always explicitly stated, that molecules having similar biological activity as wild-type or purified co-stimulatory molecules (e.g., recombinantly produced or muteins thereof) are intended to be used within the spirit and scope of the invention.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1I), interleukin-11 (IL-11), MIP-1I, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

A “subject” is a vertebrate, preferably a mammal, more preferably a human. Mammals include, but are not limited to, murines, simians, humans, farm animals, sport animals, and pets.

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

The present invention provides compositions comprised of a plurality of adenoviral particles complexed to an insoluble micro-platform material and methods for forming such complexes. These compositions provide physical properties particularly useful to facilitate their absorption and processing by phagocytic antigen presenting cells such as dendritic cells and macrophages. Thus the compositions of the invention provide methods for transfecting antigen presenting cells and for vaccinating a subject against a disease. The insoluble nature of the particulate compositions of the invention limits their ability to diffuse in vivo further enhancing their utility for vaccination in vivo.

Particulates of adenovirus particles immobilized or within a matrix are prepared and delivered to dendritic cells either in vivo or ex vivo to favor the uptake of adenovirus-containing particulates by phagocytosis. The advantages of the present invention are particularly apparent in the case of in vivo gene transfer to antigen presenting cells. While it would be expected that following injection into the skin, adenovirus particles would rapidly dissipate and the effective local concentration of virus available for transduction of dendritic cells in the skin would rapidly decrease over time, the probability of transducing skin dendritic cells would be greater if adenovirus particulates are administered because the reduced mobility of the adenovirus particulates would restrict their dissipation from the site of injection, increasing the exposure time of virus to dendritic cells while the presentation of adenovirus in particulate form would favor its uptake by phagocytosis.

In one aspect the present invention provides a plurality of adenovirus particles complexed to an insoluble micro-platform material. The invention envisions a variety of alternative micro-platform materials and methods for attachment of adenovirus particles to such materials. Examples of specific micro-platform materials and methods for preparing particulates of adenovirus particles using these materials are provided below.

The invention also provides particulates of adenovirus particles further comprising a cell binding ligand. Such ligands include polypeptide, polysaccharides, lipids and synthetic mimetics of such molecules that have specific affinity for a receptor on the surface of a target cell. Thus, incorporation of the cell binding ligand into the adenovirus particulates serves to enhance the attachment of the adenovirus particles to this target cell.

In another embodiment of the invention, the cell binding ligand is a ligand for a receptor on a dendritic cell. In particular embodiments of the invention this ligand is a cytokine such as GM-CSF, mannose or mannose-6-phosphate. The dendritic cell binding ligand can be incorporated into the adenovirus particulates by cross-linking with a chemical agent or antibody molecule. Alternatively, when the cell binding ligand is a polypeptide, a gene encoding a recombinant fusion protein can be constructed so that the cell binding ligand is fused to an envelope protein of the adenovirus particle and displayed on the surface of the viral particulate.

In one embodiment of the invention, particulates of adenovirus particles are formed by attaching individual adenovirus particles to a micro-platform composed of a polymeric fiber or microbead. A variety of alternative micro-platform materials, to which viral particles will adhere via covalent or non-covalent bonds, are envisioned by the present invention. Suitable matrix material include, but are not limited: to anion exchange resins such as DEAE (diethylaminoethly) ligand resin, QAE (diethyl [2hydroxypropyl] aminoethyl) ligand resin, ecteola (epichlorohydrin triethanolamine) ligand resin, and PEI (polyethyleneimine) ligand resin; cation exchange resins such as CM (carboxymethyl) ligand resin, and SP (sulfopropyl) ligand resin; and metal chelating resins such as cellulose, agarose, sebacic acid polyglactin 910, polyanhydrins and polyorthoester polymers in zinc, cadmium, copper, or nickel containing buffers. In a particular aspect of the invention particulates of adenovirus particles can be formed by mixing streptavidin-agarose covered beads with biotinylated adenovirus particles.

The adenoviral particulates of the present invention can further comprise a polynucleotide encoding an antigenic peptide operatively linked to a promoter element so that the antigenic polypeptide will be expressed within antigen presenting dendritic cells following transduction by the adenovirus particulates. A wide variety of alternative antigenic peptides are envisioned as candidates for inclusion in adenovirus vectors of the present invention. These include antigens normally produced by infectious organisms, tumor associates antigens, and antigens expressed by other pathological cells. Because of the strong capacity of dendritic cells to present antigens and induce immune responses, both B cell and cytotoxic T cell antigens can be included in the viral vectors of the present invention. Such antigenic peptides can include both MHC class I and MHC class II epitopes.

Techniques for identifying and manipulating antigenic polypeptides and the polynucleotides that encode them are well established in the art and recombinant adenovirus vectors for producing the particulates of the present invention can be constructed using standard methods as described in detail below. Thus, the present invention provides for a variety of alternative compositions comprising particulates of adenovirus particles attached to alternative micro-platform materials, where the adenovirus vectors further comprise genes for various antigenic peptides and the particulates may further comprise ligands for receptors on the surface of dendritic cells.

The present invention further provides a method of forming a particulate of adenovirus particles comprising mixing adenovirus particles with an insoluble micro-platform material so that the adenovirus particles become complexed to the micro-platform material. In a particular embodiment of the invention the micro-platform material is a polymeric fiber or microbead that is complexed with the adenovirus particles by a crosslinking agent.

In one aspect the present invention provides a method for forming particulates of adenovirus particles by crosslinking the micro-platform and virus particles using a crosslinking agent. For example, the method of forming particulates can employ an antibody molecule to attach the viruses to the micro-platform. The antibody can be naturally occurring or engineered, such as a bifunctional or bivalent antibody.

Methods for generating polyclonal and monoclonal antibodies that would bind specifically to the adenovirus particles of the invention have been demonstrated in the art and are describe in further detail below. Antibodies have been widely used to conjugate polypeptides to support materials and techniques for attaching the adenovirus particles to the support materials of the present invention can be adapted without undue experimentation by individuals of skill in the art. For example, an anti-adenovirus monoclonal antibody can be biotinylated and then attached to a micro-platform material that contains streptavidin, such as streptavidin agarose beads. The materials and reagents required for accomplishing such methods are commonly available from many suppliers.

In a separate embodiment of the invention, the method of forming particulates of adenovirus particles can comprise selecting a micro-platform material to which adenovirus particles will attach spontaneously via covalent or non-covalent bonds such as hydrophobic, hydrophilic, ionic or electrostatic bonds. A number of different anionic, cationic and metal chelating resins are useful for performing this method. For example, adenovirus particles can be attached to the anionic DEAE (diethylaminoethyl) resin at physiologic pH by simply mixing appropriate concentrations of virus particles with the resin at room temperature and allowing the virus particles to spontaneously attach to the resin. Similarly, adenovirus particles will rapidly and spontaneously attach to the cationic CM (carboxymethyl) resin at pH near seven. Furthermore, adenovirus particles have a high affinity for metal chelating resins such as cellulose, dextran and agarose when the viruses are mixed with the resins in the presence of zinc, cadmium, copper or nickel cations.

The methods of the invention can also comprise addition of a ligand for a cell surface receptor present on the surface of dendritic cells. Thus, the particulates of adenovirus particles can be produced so as to incorporate a polypeptide ligand for a dendritic cell receptor such as GM-CSF or a polysaccharide ligand such as mannose or mannose-6-phosphate.

The present invention further provides a method of transfecting a dendritic cell comprising contacting a dendritic cell with a particulate of adenovirus particles, thereby transfecting the dendritic cells. Dendritic cells have a natural ability to ingest particulate material by the process of phagocytosis. This invention takes advantage of this capacity by presenting the dendritic cell with an insoluble material easily assimilated by this process.

The transfection method of the invention is intended to be performed both in vivo and ex vivo. For ex vivo administration dendritic cells can be isolated or generated using methods described in detail below. Once pulsed with antigen, the transfected dendritic cells can either be administered directly to a subject or further used in vitro to present antigens to immune effector cells such as cytotoxic T cells. T cells educated by exposure to dendritic cell presented antigens are then useful for adoptive immunotherapy.

The adenovirus particulates of the invention can also be administered in vivo using standard methods for vaccination. Thus the particulates can be injected dermally, intravenously, intramuscularly, intranasally, or intraperitoneally. In a preferred embodiment the particulates of adenovirus particles are administered dermally. Finally, the particulates of adenovirus particles of the invention can be formulated for delivery as a vaccine using a variety of alternative pharmaceutically acceptable carriers as described in further detail below and administered with appropriate adjuvants to stimulate an immune response.

Construction of Recombinant Adenoviral Vectors

Adenovirus vectors useful in the genetic modifications of this invention may be produced according to methods already taught in the art. (see, e.g., Karlsson et al. (1986) EMBO J. 5:2377; Carter (1992) Curr. Op. Biotechnol. 3:533-539; Muzcyzka (1992) Current Top. Microbiol. Immunol. 158:97-129; and GENE TARGETING: A PRACTICAL APPROACH (1992) ed. A. L. Joyner, Oxford University Press, NY). Several different approaches are feasible. Preferred is the helper-independent replication deficient human adenovirus system.

The recombinant adenoviral vectors based on the human adenovirus 5 (Virology 163:614-617, 1988) are missing essential early genes from the adenoviral genome (usually E1A/E1B), and are therefore unable to replicate unless grown in permissive cell lines that provide the missing gene products in trans. In place of the missing adenoviral genomic sequences, a transgene of interest can be cloned and expressed in cells infected with the replication deficient adenovirus. Although adenovirus-based gene transfer does not result in integration of the transgene into the host genome (less than 0.1% adenovirus-mediated transfections result in transgene incorporation into host DNA), and therefore is not stable, adenoviral vectors can be propagated in high titer and transfect non-replicating cells. Human 293 cells, which are human embryonic kidney cells transformed with adenovirus E1A/E1B genes, typify useful permissive cell lines and are commercially available from the ATCC. However, other cell lines which allow replication-deficient adenoviral vectors to propagate therein can be used, including HeLa cells.

Additional references describing adenovirus vectors which could be used in the methods of the present invention include the following: Horwitz M. S. Adenoviridae and Their Replication, in Fields B. et al. (eds.) VIROLOGY, Vol. 2, Raven Press New York, pp. 1679-1721, 1990); Graham F. et al. pp. 109-128 in METHODS IN MOLECULAR BIOLOGY, Vol. 7: GENE TRANSFER AND EXPRESSION PROTOCOLS, Murray E. (ed.), Humana Press, Clifton, N.J. (1991); Miller N. et al. (1995) FASEB Journal 9:190-199 Schreier H. (1994) Pharmaceutica Acta Helvetiae 68:145-159; Schneider and French (1993) Circulation 88:1937-1942; Curiel D. T. et al. (1992) Human Gene Therapy 3: 147-154; Graham, F. L. et al. WO 95/00655; Falck-Pedersen E. S. WO 95/16772; Denefle P. et al. WO 95/23867; Haddada H. et al. WO 94/26914; Perricaudet M. et al. WO 95/02697; and Zhang W. et al. WO 95/25071. A variety of adenovirus plasmids are also available from commercial sources, including, e.g., Microbix Biosystems of Toronto, Ontario, Canada (see, e.g., Microbix Product Information Sheet: Plasmids for Adenovirus Vector Construction, 1996). See also, the papers by Vile et al. (1997) Nature Biotech. 15:840-841 and Feng et al. (1997) Nature Biotech. 15:866-870, describing the construction and use of adeno-retroviral chimeric vectors that can be employed for genetic modifications.

Generation of adenovirus particulates

Particulates of adenovirus particles can be prepared using a variety of alternative methods and materials to attach adenovirus particles to a micro-platform material comprising a solid support. In one aspect of the invention particulates of adenovirus particles can be prepared by cross linking adenoviral particles with either an antibody, which can be naturally occurring or engineered such as a bifunctional antibody, or a bifunctional agent or a polymer that can bind via covalent or non-covalent (hydrophobic, hydrophilic, ionic, or electrostatic attraction) bonds.

Laboratory methods for producing polyclonal antibodies and monoclonal antibodies, as well as deducing their corresponding nucleic acid sequences, are known in the art, see Harlow and Lane (1988) Supra and Sambrook, et al. (1989) Supra. The monoclonal antibodies of this invention can be biologically produced by introducing protein or a fragment thereof into an animal, e.g., a mouse or a rabbit. The antibody producing cells in the animal are isolated and fused with myeloma cells or heteromyeloma cells to produce hybrid cells or hybridomas. Accordingly, the hybridoma cells producing the monoclonal antibodies of this invention also are provided.

Thus, using the protein or fragment thereof, and well known methods, one of skill in the art can produce and screen the hybridoma cells and antibodies of this invention for antibodies having the ability to bind the proteins or polypeptides.

If a monoclonal antibody being tested binds with the protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

Particulates of adenovirus particles can be produced by linking adenoviral particles with a micro-platform material using anti-adenovirus antibodies as a cross-linking agent. Anti-adenovirus monoclonal or polyclonal antibodies can be bound to many different carriers. Thus, this invention also provides compositions containing the antibodies and another substance, active or inert. Examples of well-known carriers include, but are not limited to glass, polystyrene, polypropylene, polyethylene, dextran, nylon, amylases, natural and modified celluloses, polyacrylamides, agaroses and magnetite. Those skilled in the art will know of other suitable carriers for binding monoclonal or polyclonal antibodies, or will be able to ascertain such, using routine experimentation.

In a separate aspect of the invention, particulates of adenovirus particles can be prepared by mixing adenovirus particles with a polymeric matrix, such as fibers or microbeads, to which viral particles will adhere via covalent or non-covalent bonds. Suitable matrix material include, but are not limited to anion exchange resins such as DEAE (diethylaminoethly) ligand resin, QAE (diethyl[2-hydroxypropyl]aminoethyl) ligand resin, ecteola (epichlorohydrin triethanolamine) ligand resin, and PEI (polyethyleneimine) ligand resin; cation exchange resins such as CM (carboxymethyl) ligand resin, and SP (sulfopropyl) ligand resin; and metal chelating resins such as cellulose, agarose, sebacic acid polyglactin 910, polyanhydrins and polyorthoester polymers in zinc, cadmium, copper, or nickel containing buffers. In a particular aspect of the invention particulates of adenovirus particles can be formed by mixing streptavidin-agarose covered beads with biotinylated adenovirus particles.

Adenovirus particles are bound spontaneously to anion exchange resins in 400 mM NaCl containing buffer by suspending the virus particles and resin in buffer solution. Particulates of adenoviral particles are then collected by centrifugation or filtration. Fractogel DEAE resin, a material comprising a tentacle bound ligand on a polymethacrylate bead may also be employed.

To form adenoviral particulates with DEAE resin, prepare gradient purified adenovirus and dilute 1:2 with PBS solution, then add particles of DEAE resin. Binding of virus to the resin is very rapid under these conditions and will be accomplished within seconds of contact. The volume of DEAE resin required to achieve a particular particle loading of viral particles can be measure empirically by performing the binding reaction with a series of concentrations of virus and resin and then quantitating the number of virus particles bound using a quantitative viral assay such as an ELISA or quantitative PCR reaction. The particulates of adenoviral particles are collected by centrifugation and resuspended in PBS buffer.

In an alternative embodiment of the invention, stable particulates of adenovirus particles can be produced with cationic resins spontaneously in neutral solutions. Similar empirical measurements of viral loading per particle are accomplished with quantitative techniques such as ELISA and PCR.

In a further embodiment of the invention adenovirus particles are bound to metal chelating resins in zinc, cadmium, copper, or nickel containing buffers. Both tentacle and non-tentacle resins of various compositions and sizes may be used. Specific resin materials include, but are not limited to, cellulose, agarose, sebacic acid, polyglactin 910, polyanhydrins and polyorthoester polymers. Viral particles are bound spontaneous to the resin at room temperature in buffered solutions containing the appropriate metal cations. Increasing concentrations of virus per particle are achieved by increasing the concentration of virus with respect to resin in the reaction mixture. Particle loading is determined by quantitative analysis using ELISA or Q-PCR techniques.

In another aspect of the invention, the particulates of adenoviral particles further comprise a ligand with specificity for a receptor on the surface of a dendritic cell. Such ligands include but are not limited to cytokines such as GM-CSF, mannose, or mannose-6-phosphate. Such ligands are incorporated into the particulates of adenovirus particles using various methods well known in the art such as chemical cross-linking agents, bivalent antibody molecules, or non-covalent attachment to the resin surface by hydrophobic, hydrophilic, ionic or electrostatic bonding. Alternatively, a gene encoding an exposed viral envelop protein can be genetically modified to encode a fusion polypeptide comprising a peptide ligand for a dendritic cell receptor so that the fusion protein is expressed on the outer surface of the viral particle.

Isolation, Culturing and Expansion of APCs, Including Dendritic Cells

The compositions of the present invention can be delivered to APCs and in particular dendritic cells, ex vivo or in vitro to pulse the APCs with adenovirus encoded antigenic polypeptide. The following is a brief description of two fundamental approaches for the isolation of APCs. These approaches involve (1) isolating bone marrow precursor cells (CD34⁺) from blood and stimulating them to differentiate into APCs; or (2) collecting the precommitted APCs from peripheral blood. In the first approach, the patient must be treated with cytokines such as GM-CSF to boost the number of circulating CD34⁺ stem cells in the peripheral blood.

Various methods to isolate and characterize APCs including DCs have been known in the art. At least two methods have been used for the generation of human dendritic cells from hematopoietic precursor cells in peripheral blood or bone marrow. One approach utilizes the rare CD34+ precursor cells and stimulate them with GM-CSF plus TNF-α. The other method makes use of the more abundant CD34− precursor population, such as adherent peripheral blood monocytes, and stimulate them with GM-CSF plus IL-4 (see, for example, Sallusto et al. (1994), Supra).

In one aspect of the invention, the method described in Romani et al (1996), (insert citation) and Bender et al (1996), J. Immunol. Methods 196:121-135, is used to generate both immature and mature dendritic cells from the peripheral blood mononuclear cells (PBMC) of a mammal, such as a murine, simian or human. Briefly, isolated PBMC are pre-treated to deplete T- and B-cells by means of an immunomagnetic technique. Lymphocyte-depleted PBMC are then cultured for 7 days in RPMI medium, supplemented with 1% autologous human plasma and GM-CSF/IL-4, to generate dendritic cells. Dendritic cells are nonadherence as opposed to their monocyte progenitor. Thus, on day 7, non-adherent cells are harvested for further processing.

The dendritic cells derived from PBMC in the presence of GM-CSF and IL-4 are immature, in that they can lose the nonadherence property and revert back to macrophage cell fate if the cytokine stimuli are removed from the culture. The dendritic cells in an immature state are very effective in processing native protein antigens for the MHC class II restricted pathway (Romani et al. (1989) J. Exp. Med. 169:1169).

Further maturation of cultured dendritic cells is accomplished by culturing for 3 days in a macrophage-conditioned medium (CM), which contains the necessary maturation factors. Mature dendritic cells are less able to capture new proteins for presentation but are much better at stimulating resting T cells (both CD4+ and CD8+) to grow and differentiate.

Mature dendritic cells can be identified by their change in morphology, such as the formation of more motile cytoplasmic processes; by their nonadherence; by the presence of at least one of the following markers: CD83, CD68, HLA-DR or CD86; or by the loss of Fc receptors such as CD115 (reviewed in Steinman (1991) Annu. Rev. Immunol. 9:271.)

The second approach for isolating APCs is to collect the relatively large numbers of precommitted APCs already circulating in the blood. Previous techniques for isolating committed APCs from human peripheral blood have involved combinations of physical procedures such as metrizamide gradients and adherence/nonadherence steps (Freudenthal, P. S. et al. (1990) PNAS 87:7698-7702); Percoll gradient separations (Mehta-Damani, et al. (1994) J. Immunol. 153:996-1003); and fluorescence activated cell sorting techniques (Thomas, R. et al. (1993) J. Immunol. 151:6840-52).

One technique for separating large numbers of cells from one another is known as countercurrent centrifugal elutriation (CCE). In this technique, cells are subject to simultaneous centrifugation and a washout stream of buffer which is constantly increasing in flow rate. The constantly increasing countercurrent flow of buffer leads to fractional cell separations that are largely based on cell size.

In one aspect of the invention, the APC are precommitted or mature dendritic cells which can be isolated from the white blood cell fraction of a mammal, such as a murine, simian or a human (See, e.g., WO 96/23060). The white blood cell fraction can be from the peripheral blood of the mammal. This method includes the following steps: (a) providing a white blood cell fraction obtained from a mammalian source by methods known in the art such as leukapheresis; (b) separating the white blood cell fraction of step (a) into four or more subfractions by countercurrent centrifugal elutriation, (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c), and (e) collecting the enriched fraction of step (d), preferably at about 4° C. One way to identify the dendritic cell-enriched fraction is by fluorescence-activated cell sorting. The white blood cell fraction can be treated with calcium ionophore in the presence of other cytokines, such as recombinant (rh) rhIL-12, rhGM-CSF, or rhIL-4. The cells of the white blood cell fraction can be washed in buffer and suspended in Ca⁺⁺/Mg⁺⁺ free media prior to the separating step. The white blood cell fraction can be obtained by leukapheresis. The dendritic cells can be identified by the presence of at least one of the following markers: HLA-DR, HLA-DQ, or B7.2, and the simultaneous absence of the following markers: CD3, CD14, CD16, 56, 57, and CD 19, 20. Monoclonal antibodies specific to these cell surface markers are commercially available.

More specifically, the method requires collecting an enriched collection of white cells and platelets from leukapheresis that is then further fractionated by countercurrent centrifugal elutriation (CCE) (Abrahamsen, T. G. et al. (1991) J. Clin. Apheresis. 6:48-53). Cell samples are placed in a special elutriation rotor. The rotor is then spun at a constant speed of, for example, 3000 rpm. Once the rotor has reached the desired speed, pressurized air is used to control the flow rate of cells. Cells in the elutriator are subjected to simultaneous centrifugation and a washout stream of buffer which is constantly increasing in flow rate. This results in fractional cell separations based largely but not exclusively on differences in cell size.

Quality control of APCs and more specifically DCs collection and confirmation of their successful activation in culture is dependent upon a simultaneous multi-color FACS analysis technique which monitors both monocytes and the dendritic cell subpopulation as well as possible contaminant T lymphocytes. It is based upon the fact that DCs do not express the following markers: CD3 (T cell); CD14 (monocyte); CD16, 56, 57 (NK/LAK cells); CD19, 20 (B cells). At the same time, DCs do express large quantities of HLA-DR, significant HLA-DQ and B7.2 (but little or no B7.1) at the time they are circulating in the blood (in addition they express Leu M7 and M9, myeloid markers which are also expressed by monocytes and neutrophils).

When combined with a third color reagent for analysis of dead cells, propidium iodide (PI), it is possible to make positive identification of all cell subpopulations (see Table 1):

TABLE 1
FACS analysis of fresh peripheral cell subpopulations

| | Color #1 | | |
|---|---|---|---|
| | Cocktail | Color #2 | Color #3 |
| | 3/14/16/19/20/56/57 | HLA-DR | PI |
| Live Dendritic cells | Negative | Positive | Negative |
| Live Monocytes | Positive | Positive | Negative |
| Live Neutrophils | Negative | Negative | Negative |
| Dead Cells | Variable | Variable | Positive |

Additional markers can be substituted for additional analysis:
Color #1: CD3 alone, CD14 alone, etc.; Leu M7 or Leu M9; anti-Class I, etc.
Color #2: HLA-Dq, B7.1, B7.2, CD25 (IL2r), ICAM, LFA-3, etc.

The goal of FACS analysis at the time of collection is to confirm that the DCs are enriched in the expected fractions, to monitor neutrophil contamination, and to make sure that appropriate markers are expressed. This rapid bulk collection of enriched DCs from human peripheral blood, suitable for clinical applications, is absolutely dependent on the analytic FACS technique described above for quality control. If need be, mature DCs can be immediately separated from monocytes at this point by fluorescent sorting for “cocktail negative” cells. It may not be necessary to routinely separate DCs from monocytes because, as will be detailed below, the monocytes themselves are still capable of differentiating into DCs or functional DC-like cells in culture.

Once collected, the DC rich/monocyte APC fractions (usually 150 through 190) can be pooled and cryopreserved for future use, or immediately placed in short term culture.

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium ionophore A23187, for example, at the beginning of a 24-48 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2. Furthermore this activated bulk population functions as well on a small numbers basis as a further purified.

Specific combination(s) of cytokines have been used successfully to amplify (or partially substitute) for the activation/conversion achieved with calcium ionophore: these cytokines include but are not limited to purified or recombinant (“rh”) rhGM-CSF, rhIL-2, and rhIL-4. Each cytokine when given alone is inadequate for optimal upregulation.

Transducing DCs

DCs can be transduced with particulates of adenovirus particles encoding a relevant antigenic polypeptides (Arthur, et al. (1997) J. Immunol. 159:1393-1403; Wan, et al. (1997) Human Gene Therapy 8:1355-1363; Huang, et al. (1995) J. Virol. 69:2257-2263). In vitro/ex vivo, exposure of human DCs to particulates of adenovirus particles at a multiplicity of infection (MOI) of 500 for 16-24 h in a minimal volume of serum-free medium reliably gives rise to transgene expression in 90-100% of DCs. The efficiency of transduction of DCs or other APCs can be assessed by immunofluorescence using fluorescent antibodies specific for the adenovirus encodes antigen being expressed (Kim, et al. (1997) J. Immunother. 20:276-286). Alternatively, the antibodies can be conjugated to an enzyme (e.g. HRP) giving rise to a colored product upon reaction with the substrate. The actual amount of antigenic polypeptides being expressed by the DCs can be evaluated by ELISA.

Presentation of Antigen to the APC

For purposes of immunization, particulates of adenovirus particles can be delivered in vivo, ex vivo or in vitro to antigen-presenting cells. Antigen-presenting cells (APCs) can consist of dendritic cells (DCs), monocytes/macrophages, B lymphocytes or other cell type(s) expressing the necessary MHC/co-stimulatory molecules. The methods described herein focus primarily on DCs which are the most potent, preferred APCs.

Pulsing is accomplished in vitro/ex vivo by exposing APCs to particulates of adenovirus particles further comprising gene encoding antigenic protein or peptide(s). The particulates of adenovirus particles are added as a homogenous suspension at a concentration of 1-10 plaque forming unit (pfu) per cell or 10-50 pfu per cell or 50 -100 pfu per cell, or 100-500 pfu per cell. The APCs are then incubated at 37° C. for 1-4 hours and then returned to culture medium for 24 hours. Pulsed APCs can subsequently be administered to the host via an intravenous, subcutaneous, intranasal, intramuscular or intraperitoneal route of delivery.

Expansion of Immune Effector Cells

The present invention makes use of APCs pulsed with particulates of adenovirus particles to stimulate production of an enriched population of antigen-specific immune effector cells. The antigen-specific immune effector cells are expanded at the expense of the APCs, which die in the culture. The process by which naïve immune effector cells become educated by other cells is described essentially in Coulie (1997) Molec. Med. Today 3:261-268.

The APCs prepared as described above are mixed with naive immune effector cells. Preferably, the cells may be cultured in the presence of a cytokine, for example IL2. Because dendritic cells secrete potent immunostimulatory cytokines, such as IL12, it may not be necessary to add supplemental cytokines during the first and successive rounds of expansion. In any event, the culture conditions are such that the antigen-specific immune effector cells expand (i.e. proliferate) at a much higher rate than the APCs. Multiple infusions of APCs and optional cytokines can be performed to further expand the population of antigen-specific cells.

In one embodiment, the immune effector cells are T cells. In a separate embodiment, the immune effector cells can be genetically modified by transduction with a transgene coding for example, IL-2, IL-11 or IL-13. Methods for introducing transgenes in vitro, ex vivo and in vivo are well known in the art. See Sambrook, et al. (1989) Supra.

Adoptive Immunotherapy and Vaccines

The expanded populations of antigen-specific immune effector cells of the present invention also find use in adoptive immunotherapy regimes and as vaccines.

Adoptive immunotherapy methods involve, in one aspect, administering to a subject a substantially pure population of educated, antigen-specific immune effector cells made by culturing naïve immune effector cells with APCs as described above. Preferably, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

In a further embodiment, APCs or immune effector cells are administered with an effective amount of a stimulatory cytokine, such as IL-2 or a co-stimulatory molecule.

Particulates of adenovirus particles can also be delivered in vivo with adjuvant via the intravenous, subcutaneous, intranasal, intramuscular or intraperitoneal route of delivery.

Methods for vaccinating a subject

The compositions of particulates of adenovirus particles described by the present invention can be prepared in a variety of forms for delivery as vaccines. For example, the particulates can be lyophilized to prepare a dried material, which is stable during extended storage and can be reconstituted in a liquid medium prior to administration. Alternatively the particulates can be suspended in a variety of pharmaceutically acceptable carriers. Such pharmaceutically acceptable carriers can include aqueous and non-aqueous isotonic solutions such as phosphate buffered saline and glucose solutions and inactivated serum. The carrier can include anti-oxidants, buffers, and bacteriostats that render the formulation isotonic as well as excipients and vaccine adjuvants. Techniques to prepare and formulate vaccines are well known in the art (reviewed in Burke, R. L. (1993) Seminars in Virology, 4:187-197.

Individuals skilled in the art will be familiar with methods for determining an effective dose of the vaccine. Administration of particulates of adenovirus particles can be achieved via different routes including intravenous, intramuscular, intranasal, intraperitoneal or cutaneous delivery. The vaccine can also be formulated for delivery through oral and ocular routes of administration. The preferred method is cutaneous delivery of adenovirus particulates at multiple sites using a total dose of approximately 1×10¹⁰-1×10¹² i.u. Levels of in vivo dendritic cell transduction can be roughly assessed by co-staining with antibodies directed against dendritic cell marker(s) and the adenovirus encoded antigen being expressed. The staining procedure can be carried out on biopsy samples from the site of administration or on cells from draining lymph nodes or other organs where DCs may have migrated (Condon. et al. (1996) Nature Med. 2:1122-1128; Wan, et al. (1997) Human Gene Therapy 8:1355-1363). The amount of antigen being expressed at the site of injection or in other organs where transduced DCs may have migrated can be evaluated by ELISA on tissue homogenates.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

More particularly, an agent of the present invention also referred to herein as the active ingredient, may be administered for therapy by any suitable route including nasal, topical (including transdermal, aerosol, buccal and sublingual), parental (including subcutaneous, intramuscular, intravenous and intradermal) and pulmonary. It will also be appreciated that the preferred route will vary with the condition and age of the recipient, and the disease being treated.

It is to be understood that while the invention has been described in conjunction with the above embodiments, that the foregoing description and the following examples are intended to illustrate and not limit the scope of the invention. For example, any of the above-noted compositions and/or methods can be combined with known therapies or compositions. Other aspects, advantages and modifications within the scope of the invention will be apparent to those skilled in the art to which the invention pertains.

1. An adenovirus particulate comprising a plurality of adenovirus particles complexed to an insoluble micro-platform material.

2. The adenovirus particulate of claim 1 further comprising a cell binding ligand complexed to the micro-platform material.

3. The adenovirus particulate of claim 2 wherein the cell binding ligand binds to a receptor on a dendritic cell.

4. The adenovirus particulate of claim 3 wherein the cell binding ligand is selected from the group consisting of GM-CSF, mannose, and mannose-6-phosphate.

5. The adenovirus particulate of claim 1 wherein the micro-platform material is a polymeric fiber or microbead.

6. The adenovirus particulate of claim 5 wherein the adenovirus particulate further comprises a gene encoding an antigenic polypeptide.

7. A method of forming a particulate composed of adenovirus particles comprising mixing adenovirus particles with an insoluble micro-platform material so that the adenovirus particles become complexed to the micro-platform material.

8. The method of claim 7 where the micro-platform material is a polymeric fiber or microbead.

9. The method of claim 7 wherein the adenovirus particles are complexed to the micro-platform material by a crosslinking agent.

10. The method of claim 8 wherein the adenovirus particles are complexed to the micro-platform material by a crosslinking agent.

11. The method of claim 9 where the cross-linking substance is a bivalent antibody.

12. The method of claim 10 where the cross-linking substance is a bivalent antibody

13. A method of forming a particulate of adenovirus particles where the adenovirus particle further comprises a gene encoding an antigenic poylpeptide.

14. The method of claim 7 wherein the particulate of adenovirus particles further comprises a ligand that binds to a receptor on a dendritic cell.

15. The method of claim 14 wherein the ligand is GM-CSF, mannose, or mannose-6-phosphate.

16. The method of claim 13 wherein the particulate of adenovirus particles further comprises a ligand that binds to a receptor on a dendritic cell.

17. The method of claim 16 wherein the ligand is GM-CSF, mannose, or mannose-6-phosphate.

18. A method of transfecting a dendritic cell comprising contacting a dendritic cell with an adenovirus particulate of claim 1, thereby transfecting the cell.

19. A method of vaccinating a subject against a disease comprising administering to the subject an adenovirus particulate of claim 6, thereby vaccinating the subject against a disease.

20. A method of claim 19 where the adenovirus particulate vaccine is administered together with an adjuvant.
